(19) United States
(12) Patent Application Publication
(10) Pub. No.: US 2011/0045096 A1
(43) Pub. Date: Feb. 24, 2011

(54) Solubilized delivery system for topical anesthetics

(76) Inventor: Pankaj Modi, Harbour Beach, MI (US)

Correspondence Address: JAMES D. FORNARI;C/O GERSTEN SAVAGE LLP, 600 LEXINGTON AVENUE, NEW YORK, NY, 10022, US

(21) Appl. No.: 12/583,366

(22) Filed: Aug. 19, 2009

(51) Int. Cl. A61K 9/51; A61K 31/167; A61P 25/04

(52) U.S. Cl. 424/498; 514/626; 977/906

(57) ABSTRACT

A nano particle drug delivery system comprised of micelles coated with lipid molecules for the non-invasive deployment and absorption of active anesthetic compounds through the stratum corneum and throughout the skin and sub cutaneous tissue without any cutaneous toxicity.

### BACKGROUND OF THE INVENTION

Anesthesia is a process commonly used to block the perception of pain. Topical anesthetics are widely used agents that are absorbed by the skin and temporarily block nerve endings that perceive inflammation and other skin injury. Injected general anesthetics are usually described as ones which may be injected to provide diffuse pain blockage over areas greater than the injection site as distinguished from local anesthetics which merely block pain in the applied area and are not injected.

Injected anesthetic agents are often used in procedures carried out on various tissues and organs. For example, with regard to procedures performed on the eye, common anesthetic agents utilized include subconjunctival injections of aqueous lidocaine and tetracaine drops. However, subconjunctival injections of aqueous lidocaine are less than desirable as many patients suffer from anxiety caused by needle phobia and/or the physical pain caused by the actual injection. Indeed, it is believed that the anxiety levels can reach the point where patients avoid the necessary medical care. The topical or local administration of tetracaine drops avoids these needle-related problems. However, there are some drawbacks with such drops. Some of the drops administered to patient may miss the eye due to the shaking of the hand or the blinking of the eye. The residence time of the drop on the eye is limited, for example, less than about a minute. Thus, the anesthetic efficacy of the tetracaine drops could become insufficient since both the onset of anesthesia is not rapid, and the duration of anesthetic activity is limited.

Local anesthetics cause loss of feeling before and during surgery, dental procedures (including dental surgery), or labor and delivery. These medicines do not cause loss of consciousness. Additionally, local anesthetics can be used to numb any topical pain such as an irritation, burn, scrape, cut, or insect bite.

Before performing dermatological treatments, a patient is often locally anesthetized with topical anesthetics. Existing topical anesthetics used on the face or other parts of the body take up to an hour or more to anesthetize effectively and often have to be used in conjunction with occlusive dressings to enhance penetration of the anesthetic. The delay between application and effective anesthesia causes waiting room delays in a medical office and the use of the dressing and related viscous semi-liquid carries can often result in inconvenience and possible irritation and non-sterile environments.

Attempts have been made to provide a topical anesthetic in various forms, but the delivery of the anesthetic has often been difficult as well as labor and time intensive. Also, because of the different absorption rates for individuals, the actual time necessary to achieve adequate numbness is often an unknown variable which can delay procedures. This variability in length of onset time leads to delays in the commencement of medical procedures and, because of the very wide variation in onset time, can lead to the premature commencement of procedures, thereby inflicting unnecessary pain on the patient.

Various delivery systems have been proposed and utilized, but each has inherent difficulties in administration and in the actual onset time for the numbing of the applicable area.

One form of an anesthetic formulation which was proposed for use in eye procedures is described in U.S. Patent Application No. 2009/0123527 by ALAM which discloses an aqueous gel formulation comprising water, an anesthetic, a viscoelastic polymer, and a tonicity modifier. The formulation may also contain a pH adjusting agent or a product produced as a result of pH adjustment. The aqueous gel formulation is targeted for application to various tissues or organs (internal or external) of an animal, particularly to the eye of a human for inducing topical anesthesia to a tissue or organ of an animal.

The ALAM application further describes a method of inducing topical anesthesia in a tissue or organ of an animal comprising: a) providing an aqueous gel formulation comprising water, an anesthetic, a viscoelastic polymer, and a tonicity modifier, wherein the anesthetic is present in an amount of 15 mg per ml to about 50 mg per ml of the formulation, and the gel formulation is free of preservatives and phosphate buffer, is isotonic with physiological fluids, and is sterile having less than about 100 particles of 50 microns particle size or more per ml of the aqueous gel formulation; and b) topically administering an effective amount of the aqueous gel formulation to the tissue or organ of the animal. The ALAM application does not disclose any nano particle delivery system and employs a viscous material to deliver the anesthetic.

By way of further example, U.S. Patent Application No. 2006/0067958 by VALENCIA describes a delivery system which employs alcohol to lead to faster onset of action and enhanced efficacy of pharmaceutical actives. The delivery system consists of topical alcoholic gel compositions containing dissolved actives. The compositions contain less than 20% w/w water, and use hydroxypropylcellulose and non-neutralized, partially neutralized, or fully neutralized acrylic acid-based polymer as synergistic gelling agents, and diols and/or triols as an optional tertiary synergistic gelling agent. With the gelling system, the amount of base used to neutralize the acrylic acid-based polymer can be reduced or eliminated altogether. In addition, if fully neutralized acrylic acid-based polymer is used, a lower level of this polymer is required when used with the gelling system of the present invention. Finally, the gelling system is said to allow one to achieve higher viscosity in an alcoholic gel than what is possible with the fully neutralized acrylic acid-based polymer approach of prior art. Again, the system does not provide for a truly penetrating delivery system which encapsulates the active material, but rather only shows a gel system to keep the active ingredients in place and permits higher viscosity.

Another use of alcohols to decrease the time before numbness sets in is described in U.S. Patent Applications Nos. 2008/0176948 and 2009/0048347 by COHEN which describes a topical anesthetic for rapid local anesthesia. The topical anesthetic includes an anesthetic, a volatile solvent, and a non-volatile solvent. The non-volatile solvent system includes oleyl alcohol and propylene glycol. Generally, the fatty alcohol can be a C.sub.10 to C.sub.14 saturated alcohol, a liquid-at-room-temperature C.sub.12 to C.sub.22 mono- or polyunsaturatured or branched chain alcohol, or those same compounds in acid form. The fatty alcohol forms two to six percent of the formulation by weight, and, in particular, four percent by weight of the formulation. The other non-volatile solvent, propylene glycol or a butane diol with adjacent hydroxyl groups, forms between two and six percent by weight of the formulation.

The formulation includes a volatile, short-chain alcohol such as isopropyl alcohol (IPA) or ethanol. Short-chain alcohols include the isomers of butanol, propanol, ethanol, and methanol. The short-chain alcohol forms between sixty and eighty-five percent by weight of the formulation. A thickener can be added that is soluble in the total solvent system. A suitable thickener is hydroxypropylcellulose (HPC). The thickener can form between two and five tenths and three and five tenths percent by weight of the formulation. The HPC is sold under the trade name KLUCEL.

Other systems are:

U.S. Pat. No. 7,273,887 by Wepfer which discloses a topical anesthetic formulation which is typically a solution that preferably includes lidocaine, as the active anesthetic ingredient with benzyl alcohol and isopropyl alcohol. However, the solution is difficult to apply and as it rolls to other near by sites and usually spills and is dangerous to children where they can rub their hands and touch other parts of body e.g., mouth or eyes etc and may invite danger to their life.
U.S. Pat. No. 5,447,930 by Nayak which discloses a topical anesthetic for the relief of various skin irritations such as minor burns, insect bites, rashes and allergic reactions. It is prepared using pramoxine hydrochloride and zinc acetate as the active ingredients and benzyl alcohol as solvent.
U.S. Pat. No. 5,013,545 to Blackmon et. al. discloses aqueous gel-containing topical medications comprising high concentrations of alcohol, water and topically effective amounts of a pharmaceutical active such as hydrocortisone, diphenhydramine hydrochloride, lidocaine or miconazale nitrate in a gel matrix primarily consisting of water-soluble carboxyvinyl polymers. A gel clarifying agent may be optionally added for aesthetic reasons.
U.S. Pat. No. 4,937,078 to Mezie et. al. discloses the incorporation of certain concentrations of topical anesthetic actives into liposomes which are of a substantially greater size than nano particles.
U.S. Pat. No. 5,081,158 to Pomerantz discloses the use of medicated protective films as a carrier for topical anesthetics. The films are comprised of hydroxypropyl cellulose (HPC) and an esterification agent which renders the HPC soluble in a non-volatile solvent such as ethanol, isopropanol or methanol. Medicinal compounds such as benzocaine, dyclonine hydrochloride and a variety of other topical anesthetics, antibiotics and steroids are incorporated which, when applied to the skin, result in situ formed medicated films from which the actives are released to provide a sustained supply of the medicine at the treatment site.
U.S. Pat. No. 5,002,974 to Geria discloses a topical anesthetic and skin moisturizing composition comprising any one of a number of topical anesthetics, including pramoxine, in an oil-in-water emulsion including a dissolved surface active agent. The composition is asserted to provide an aesthetically pleasing analgesic skin care product. The emulsion not only provides relief from the pain associated with irritated skin but is asserted to soften and moisturize the skin with an oily coating.
U.S. Pat. No. 4,493,591 to Fourman et al discloses skin care cosmetic formulations comprised of a cellulosic polymer/solvent system capable of dispersing thin, substantive films upon the skin. Such films may serve as a carrier for sun blocking agents and insect repellents and also serve to prevent water loss form the skin surface to the environment. Finally, U.S. Pat. No. 4,389,418 to Burton et. al., in a more general and traditional sense, discloses the use of hydrocarbons such as petrolatum, paraffin wax and ozokerite and other emollients as skin moisturizing materials. These function by covering the skin with a hydrophobic occlusive film which prevents water loss from the skin to the environment.

One particular topical anesthetic utilized to suppress or eliminate pain during such procedures is known by the trade name EMLA. EMLA has a very long onset time, which is the time between administration of the topical anesthetic and the commencement of the anesthetic effect. It must also be covered with an occlusive dressing to enhance penetration. The onset time for EMLA. can range from 45 to 90 minutes and, in some instances, can take even longer. The variability in length of onset time leads to delays in the commencement of medical procedures and, because of the very wide variation in onset time, can lead to the premature commencement of procedures, thereby inflicting unnecessary pain on the patient.

Another particular topical anesthetic which may be obtained over-the-counter is known by the trade name LMX 4, which contains 4% Lidocaine cream. The application and onset time is described by the Cincinnati Children's Hospital as follows:

How does L.M.X.4 work?
At the nerve endings, L.M.X.4 causes numbing of the skin and surrounding tissue.
How do I apply L.M.X.4?
DO NOT CLEANSE THE SKIN prior to L.M.X.4 application (although you should not apply over moisturizers or other topical medications).
L.M.X.4 works best when it mixes with the skin surface oils.
Begin by rubbing a small amount of L.M.X.4 cream into each of the sites for ˜30 seconds. Make sure you are wearing gloves.
Follow this application with a thicker coating of L.M.X.4 cream. Cover cream with an occlusive dressing (ex. Tegaderm or round Band-aids).
Although L.M.X.4 does not require the use of an occlusive dressing, one is recommended for use in children to ensure adequate contact of the cream with the skin and to prevent the accidental ingestion of the cream.
Do NOT flatten the cream (L.M.X.4 works best when it is applied thickly).
After the product is removed, the skin should be cleaned and prepared as usual for the procedure.
   How long does it take for L.M.X.4 to work?
   L.M.X.4 starts to work in ˜30 minutes.
   Total application time should usually not exceed 60 minutes (rubbing it in longer has not been shown to increase its effect).

Thus, none of the existing art provided all of the advantages and benefits of the present invention. It would be advantageous and desirable to develop a topical anesthetic formulation which has a shorter onset time, which has less variability in the onset time, does not require occlusion, is easier to apply with less mess and which is amenable to use for cutaneous laser procedures such as hair removal and skin resurfacing, as well as for use before giving injections, starting IVs, drawing blood, biopsies and minor superficial surgeries. Such a formulation will have a potent clinical use with a more rapid onset of action.

### SUMMARY OF THE INVENTION

A nano particle drug delivery system comprised of micelles coated with lipid molecules for the non-invasive deployment and absorption of active anesthetic compounds through the stratum corneum and throughout the skin and sub cutaneous tissue without any cutaneous toxicity. The drug delivery system comprises a local anesthetic compound such as Lidocaine in an amount of 4% by weight and a formulation composed of nano particle micelles made out of the absorption enhancers which encapsulate the local anesthetic compound and transports it into the deep layers of the skin without causing any toxic reactions and damage to the skin layers. These micelles are very fragile and highly biodegradable thus releasing the materials substantially instantaneously on penetration giving rapid numbness.

In certain embodiments, the anesthetics such as Lidocaine alone or in combination with Benzocaine or Tetracaine or all 3 in one formulation may be employed. Furthermore, this drug delivery system allows cream or gel formulation to be employed with superior delivery requiring NO occlusion and shorter waiting time (acts within 3-10 min or less) and are easier to apply with less mess. The various formulations are completely absorbed after the application leaving no residue or greasy feeling and are not spreadable to other parts of the body but are more localized to the treatment area.

### BRIEF DESCRIPTION OF THE DRAWING

FIG. 1 is a graphical representation of the relative pain experienced by individuals where an embodiment of the invention was employed to apply the topical anesthetic.

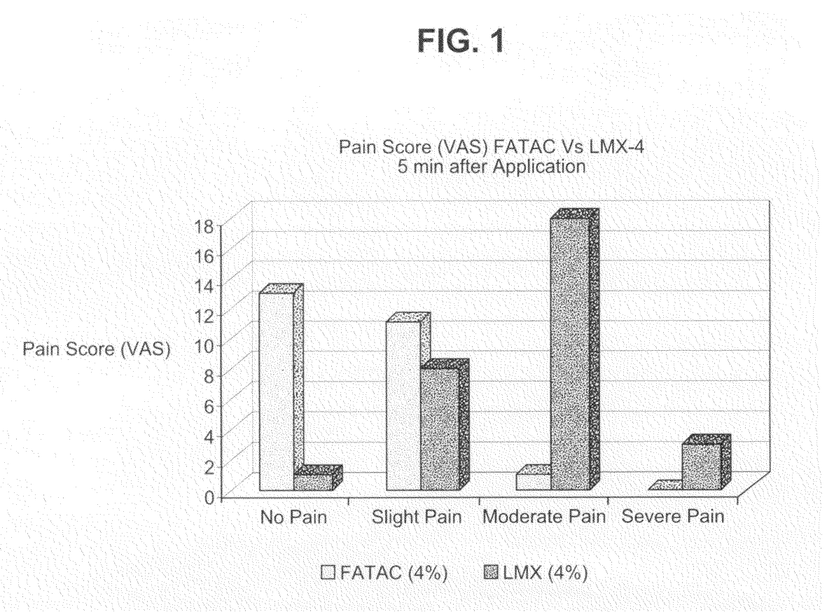

### DETAILED DESCRIPTION OF THE INVENTION

### Definitions

In describing and claiming the present invention, the following terminology will be used in accordance with the definitions set forth below.

The singular forms “a,” “an,” and “the” include plural referents unless the context clearly dictates otherwise. Thus, for example, reference to “a solvent” includes reference to one or more of such solvents, and reference to “the dispersant” includes reference to one or more of such dispersants.

As used herein, “formulation” and “composition” may be used interchangeably and refer to a combination of elements that is presented together for a given purpose. Such terms are well known to those of ordinary skill in the art.

As used herein, “carrier,” “inert carrier,” and “acceptable carrier” may be used interchangeably and refer to a carrier which may be combined with a one or a plurality of agents in order to provide a desired composition. Those of ordinary skill in the art will recognize a number of carriers that are well known for making specific remedial compositions.

As used herein, “biologically acceptable carrier” refers to a material which is suitable for use in connection with a particular biological material. A biologically acceptable carrier is compatible with, and does not adversely affect, a biological material or subject contacted therewith under prescribed conditions.

As used herein, “cosmetic” is an adjective referring to improving the appearance of a surface or covering defects. Typically, cosmetic compositions can be used to improve aesthetic rather than functional aspects of a surface. Most commonly, cosmetic compositions are formulated for application as a beauty treatment or for affecting personal appearance of the body, for example, natural tooth enamel and dental veneer surfaces.

As used herein, “remedial” is an adjective referring to remedying, correcting, treating, improving, or preventing an undesirable condition. A remedial composition can therefore be formulated to remove undesirable stains from the surface of natural tooth enamel or veneer. Similarly, remedial compositions can be configured to remove, prevent or minimize formation of undesirable elements such as stain build up and the like.

As used herein, “biological material” refers to any material which is a product of a biological organism. Typical biological materials of interest can include organic oils and the like.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of 1 to 5 should be interpreted to include not only the explicitly recited limits of 1 and 5, but also to include individual values such as 2, 2.7, 3.6, 4.2, and sub-ranges such as 1-2.5, 1.8-3.2, 2.6-4.9, etc. This interpretation should apply regardless of the breadth of the range or the characteristic being described, and also applies to open-ended ranges reciting only one end point, such as “greater than 25,” or “less than 10”.

The term “volatile component” as used herein refers to a component (e.g., a solvent or combination of solvents) that changes readily from solid or liquid to a vapor, e.g., that evaporates readily at some temperature at or below body temperature and less readily at room temperature, such as a component that evaporates rapidly between 21 and 37.degree. C. at atmospheric pressure.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms of the disease, disorder or condition, i.e., causing regression of the disease, disorder and/or condition.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the term “saturation” refers to the point at which a solution of a substance (e.g., a local anesthetic agent) can dissolve no more of that substance and additional amounts of it will appear as a precipitate. The phrase “near saturation” refers to a solution which is at least 90% saturated, such as 90%, 91%, 92%, 93%, 94%, 95%, 96%, 97%, 98%, or 99% saturated. The phrase “above saturation” refers to a solution which has a higher concentration of substance (e.g., a local anesthetic agent) than the concentration at which the solution is saturated (e.g., it is greater than 100% saturated).

The drug delivery system and methods of the present invention may be utilized to treat an individual in need thereof. In certain embodiments, the individual is a mammal such as a human, or a non-human mammal

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The drug delivery system of the present invention can be administered to a subject topically, for example, as a gel, foam, solution, lotion, cream, ointment or spray applied to the skin.

The drug delivery system may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the anesthetic agent which produces an anesthetic effect.

Drug delivery systems of the present invention for topical or transdermal administration include powders, sprays, ointments, pastes, creams, lotions, gels, solutions, patches and inhalants. The anesthetic agent may be mixed under sterile conditions with the other components of the drug delivery system, and with any preservatives, buffers, or propellants that may be required.

The drug delivery systems of the present invention may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like.

Actual dosage levels of the active ingredients in the drug delivery system may be varied so as to obtain an amount of the active ingredient that is effective to achieve the desired anesthetic response for a particular patient, composition, and mode of administration, without being toxic to the patient.

The selected dosage level will depend upon a variety of factors including the activity of the particular anesthetic agent or combination of anesthetic agents employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the drug delivery system required. For example, the physician or veterinarian could start doses of the drug delivery system or anesthetic agent at levels lower than that required in order to achieve the desired anesthetic effect and gradually increase the dosage until the desired effect is achieved. By “therapeutically effective amount” is meant the concentration of a local anesthetic agent that is sufficient to elicit the desired anesthetic effect. It is generally understood that the effective amount of the anesthetic agent will vary according to the weight, sex, age, and medical history of the subject. Other factors which influence the effective amount may include, but are not limited to, the severity of the patient's condition, the disorder being treated, the stability of the anesthetic agent, and, if desired, another type of anesthetic agent being administered with the anesthetic agent of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

### Exemplification

### Formulation

The mixture of anesthetics were formulated as cream or gel using the following ingredients which approved by the FDA and Health Canada for human use in North America.

The gel was prepared using the following ingredients;

### Medicinal Ingredients;

Lidocaine 4% by weight, the amount of Lidocaine or any other CAINE (e.g. tetratcaine, prilocalne, benzocaine, bupivaciane, etc) family members can vary from 1% to 25% by weight in any combination

### Non-Medicinal Ingredients;

All other excipients are non-medical ingredients and are all FDA approved GRAS listed items.
Gel contains no preservatives in view of the anti-microbial activities of Lidocaine, Tetracaine and Benzocaine.

### Procedure:

Heat Phase A and Phase B separately with agitation at 75-80 C. Add Phase A to Phase B and mix 30 minutes at 75 C. Cool down to 20-22 C and then add Phase C, D and continue to agitate until homogenous and one phase.

### Preparation of Phase-D; Lidocaine Micelles Solution;

Lidocaine is weighed accurately (4 gm or 5 gm) depending on the strength e.g. 4% wt or 5% wt. This powder was placed in the beaker equipped with high speed stirrer and hot plate to heat the solution. The powder of Lidocaine was added to the alcohol mixtures (ethyl, isopropyl, propylene glycol, cetyl and certery alcohol, and lanolin alcohol). The Lidocaine was allowed to dissolve slowly at 25 C with constant stirring. The solution was heated slowly to 30 C with continuous stirring till all Lidocaine was completely dissolved. To this solution, Phase-A was added to form micelles with constant stirring at a high speed (100 rpm). The solution was kept at 30 C and the agitated continuously until utilized i.e., added to Phase C (HA gel). The gel or viscous solution was cooled to room temperature at 20 C and then added to the mixture of Phase B with vigorous stirring at high speed (1000 rpm). The mixture was stirred for another 30 min at room temperature until uniform homogenous cream (lotion) was formed.

Wt %

Phase A:

| | |
|---|---|
| De-ionized Water | 16.5% |
| Tetra Sodium EDTA | 0.5-0.7% |
| Glycerin | 2.0% |
| DMSO | 0.5-1.0% |
| SLS (SDS) | 0.5-1.0% |
| Diazolidinyl UREA | 0.2% |
| d-limonene | 0.7% |
| Allantoin | 0.5% |
| Fulvic Acid | 0.5% |
| Quillaja saponaria (QTS) | 0.3% |
| Acanthophyllum squaimsom (ATS) | 0.3% |
| Myrrh Extract | 0.2% |

Phase B:

| | |
|---|---|
| Polysorbate-85 | 1.0% |
| Polysorbate 60 | 0.5-1% |
| HoHoba Oil | 0.5-1% |
| Lanolin | 1-2% |
| Tocopheryl Acetate | 0.5-1% |
| Dimethicone 200 | 0.7-1.0% |
| BHA | 0.1% |

Phase C:

| | |
|---|---|
| Fragrance | 0.01% |
| Aloe Vera (powder) | 1.5%-2.0% |
| CoQ-10 | 0.5% |
| Hyaluronic Acid (pure) | 1.0-1.5% |
| Talcum Powder (Ti02) | 1.0-1.5% |

Phase D:

| | | |
|---|---|---|
| Ethyl Alcohol + Isopropyl Alcohol | 60.0% | |
| 50:50 mixture | | |
| Propylene Glycol | 2.0% | |
| Cetyl Alcohol (Ado 1 52 NE) | 1-2.0% | |
| Cetearyl Alcohol | 1-2.0% | |
| Lanolin Alcohol (Ritachol) | 1.0% | |
| Lidocaine | 4.0-5.0% | |
| Total | 100% | by wt |

The utility and efficacy of the preparation was demonstrated in various test in both animals and humans.

### Animal Study

### In Vivo Assessment of Percutaneous Local Anaesthetic Preparations

### Assessment of the Efficacy of the Delivery System for Topical Anesthetic Cream

Objective; To assess the analgesic activity of fast-acting topical anesthetics cream (“FATAC”) in accordance with the teaching of the invention along with the superior delivery system in comparison with regular EMLA or Lidocaine or placebo creams using the heat or mechanically induced pain by a needle pricking in rat model.

Method; 50 male or female healthy rats were purchased and acclimatized for 5 days. Rats were shaved on their hind legs to remove hair to facilitate the absorption of the topical creams. Rats were distributed in 5 groups (12-13 rats in each group), each receiving topically one of the following: FATAC, EMLA (Eutectic Mixture of Local Anesthetics), Lidocaine and placebo base cream (controls) on four separate occasions 3-7 days apart.

The rats were treated as follows;

Treatment-1 Dermacaine cream (1 ml) or

### Treatment-2 Regular EMLA gel (1 ml) or

### Treatment-3 Lidocaine (1 ml) LMX

Treatment-4 Placebo cream (1 ml)

The rats were tested for the analgesic activity (pain reduction) by a heat treatment with the heated rod at 45-50 C and also by a needle prick using 28 gauge sterile needles at 2 min, 5 min, 10 min, 15 min, 30 min, 45 min and 60 minute after the application of the creams containing FATAC or EMLA or Lidocaine creams. The pain scores were assessed as follows;
0—No pain, paw not withdrawn or no violent retraction or movement on pricking
1—Minor pain, paw withdrawn slightly or slight retraction or twitching movement on pricking or heat treatment
2—Detectable pain, paw withdrawn more or good retraction or twitching movement on pricking or heat treatment
3—Painful symptoms, complete withdrawal or violent retraction or movement on pricking or heat induced treatment

### Results;

| Treatment | Time | | | | | | | |
|---|---|---|---|---|---|---|---|---|
| Pain score (Average) | 0 | 2 | 5 | 10 | 15 | 30 | 45 | 60 min |
| FATAC | 3 | 2 | 1 | 1-0 | 0 | 0 | 0 | 0 |
| (Delivery system) | | | | | | | | |
| Lidocaine Cream | 3 | 3 | 3 | 3 | 3 | 3 | 3-2 | 2-1 |
| (LMX) | | | | | | | | |
| EMLA cream | 3 | 3 | 3 | 3 | 3 | 3-2 | 2 | 2-1 |
| Placebo Cream | 3 | 3 | 3 | 3 | 3 | 3 | 3 | 3 |

Conclusions; All treatments containing actives produced anti-analgesic effects (reduction in the pain intensity) except the placebo treatment. The novel improved formulation with the drug delivery system produced the anti-analgesic effects much faster (within 5 min, p<0.0001) when compared with the regular EMLA cream or Lidocaine formulation. The regular EMLA formulation and/or Lidocaine produced analgesic effect in approximately 60 min. Thus, the FATAC topical formulation with a drug delivery system in accordance with the teaching of the invention could be valuable for very rapid skin penetration and thus bringing a rapid pain relief quickly within 5 min after the application.

### Human Clinical Trial;

### In Vivo Assessment of Percutaneous Local Anaesthetic Preparations

### Assessment of Efficacy of the Delivery System for FATAC

Objective; To assess the analgesic activity (reduction in pain sensation) of the fast-acting topical FATAC with the superior delivery system in healthy human subjects in comparison with the placebo cream and/or the regular available EMLA cream or Lidocaine by a needle pricking method.

Method; In this randomized, placebo-controlled, crossover trial in 40 healthy human (male or female) subjects, each gave written informed consent. The study was approved by the local Ethics Committee (St. Joseph Hospital, Toronto). Each volunteer received all formulations. Both left and right forearms (ventral surface at or below the anterior cubital fossa) were used and a minimum period of 7 days was maintained between successive applications on previously treated individuals. Formulations were allocated to volunteers on a random basis. Subjects were examined for the speed of onset of cutaneous anesthesia of the fast acting novel formulation of FATAC vs eutectic mixture of local anesthetics (EMLA) vs Lidocaine (LMX-4) or placebo cream treatments were compared on four separate occasions 3-7 days apart.

### Statistical Analysis

Each preparation was analysed initially for efficacy directly against placebo (table I) using the chi-square test. Thereafter, both preparations were compared directly, using the standard procedure, in terms of efficacy (chi-square) and, where possible, both duration and onset of effect (two-tailed unpaired t test)

Treatment 1; application of FATAC.
Treatment 2; application of EMLA cream
Treatment 3; application of Lidocaine cream
Treatment 5; Placebo cream

FATAC or EMLA cream or Lidocaine cream or gels were applied on the fore-arm of each subjects covering approximately 2 cm area. The cream was applied with a spatula with light gentle rubbing for 1 min.

The pain assessments were measured at 2 min, 5 min, 10 min, 15 min, 30 min, 45 min and 60 min. Pain was tested by pricks with a 28 g needle. Pain scores and subject's preference for the FATAC or EMLA or Lidocaine cream were measured at each time point. The pain score was assessed as follows;
0 No pain, hand not withdrawn or no violent retraction or movement on pricking
1 Minor pain, hand withdrawn slightly or slight retraction or twitching movement on pricking
2 Detectable pain, hand withdrawn more rapidly or good retraction or twitching movement on pricking
3 Painful symptoms, complete withdrawal or violent retraction or movement on pricking

### Results;

| Treatment | Time | | | | | | | |
|---|---|---|---|---|---|---|---|---|
| Pain score (Average) | 0 | 2 | 5 | 10 | 15 | 30 | 45 | 60 min |
| FATAC | 3 | 2 | 1 | 1 | 1-0 | 0 | 0 | 0 |
| (with delivery system) | | | | | | | | |
| EMLA cream | 3 | 3 | 3 | 3 | 3 | 2 | 2-1 | 1 |
| Lidocaine cream (LMX-4) | 3 | 3 | 3 | 3 | 3 | 2 | 1 | 1 |
| Placebo Cream | 3 | 3 | 3 | 3 | 3 | 3 | 3 | 3 |

Conclusions; Based on both pain scores and subject's preference, cutaneous anesthesia was achieved in very short time period (within 5 min, p<0.0007) in the FATAC treatment group as compared with EMLA or Lidocaine cream group or the placebo group at all time points. The analgesic effects were lasted approximately 1-2 hours. There were no significant adverse effects.

From a clinical viewpoint there may be a considerable advantage, under certain circumstances, in using a percutaneous local anaesthetic preparation possessing a prolonged activity. For example, in taking split-skin grafts a prolonged anaesthetic action may considerably reduce postoperative pain. Furthermore, the FATAC formulation has a demonstrably shorter onset time than EMLA, with consequent advantages for both ward routine and potential outpatient clinical usage There were NO major adverse events reported during the study period except 3 subjects had redness on their fore-arm and 4 subjects reported slight itchy sensation after the application of the creams in all groups including the placebo arm. The redness and the itchy sensations disappeared in less than 15-20 min.

Thus, the FATAC topical formulation with a novel drug delivery system could be valuable for very rapid skin penetration and thus bringing a rapid pain relief quickly within 5 min after the application. Other applications includes; treatment of burns, cuts, insect bites pain managements, minor surgical procedures (instead of using lidocaine injections), and many cutaneous cosmetics procedures requiring pre-anesthetic treatments.

### Clinical Studies for Facial Aesthetic Procedures

Minimizing Discomfort during the injection of DreamFill (HA Gel)™ with the use of FATAC

DreamFill (HA Gel)™ has been tested extensively in patients with associated lipoatrophy as well as for facial wrinkles and nasolabial folds but can be painful to inject especially in the latter area. This discomfort can be severe enough that after an injection with DreamFill (HA Gel)™, a patient, despite excellent results, may refuse additional treatments. We hereby describe several methods of minimizing discomfort during DreamFill (HA Gel)™ injections of nasolabial folds and other facial areas. Among the variety of methods used to lessen pain during DreamFill (HA Gel)™ injections are topical anesthetics, regional anesthetic blocks, and local anesthetic. We describe here our clinical findings for use of our novel fast acting topical anesthetic FATAC for reduction or complete elimination of discomforts and pain during the facial aesthetic procedure with dermal filler injections.

### Methods

A total of 20 consecutive patients were selected to participate in the study. The mean±SD patient age was 50.46±10.23 years. No participants were treated previously with any permanent injectable filler. Having a history of previous facial surgery involving the areas of treatment or previous placement of permanent alloplastic facial implants was an exclusion criterion for participation in the protocol. Skin type based on the Fitzpatrick classification was recorded for each participant. Patients of each skin type were treated in a similar manner. Skin pretesting was not performed on the participants.

Before treatment, a topical anesthetic containing 4% Lidocaine was applied, unoccluded, for 5-20 minutes to the area to be treated on left face and on the right side the LMX (4% Lidocaine) gel was applied.

The NASHA gel was injected into the superficial to middle dermal layer with the needle inserted bevel up. For linear depressions, serial punctures were combined with a linear threading technique. For broader depressions, fanning or cross-hatching techniques were used, supplemented with serial punctures. Rhytids were corrected to 100% of their depth, but without overcorrection. The maximum amount of “filler” used in a single area was 1.4 cm³. For lip augmentation, 1 or 2 techniques were used, depending on the patient's desires. Linear threading was used to augment the nasolabial folds vermilion border. The NASHA gel was then injected directly into the “body” to augment the volume of selected sections.

### CONCLUSION

Our techniques did not involved the use of regional or infraorbital nerve blocks and thus are simpler to perform for many dermatologists who do not routinely perform these kinds of blocks, yet can provide adequate anesthesia to help the practitioner inject DreamFill (HA Gel)™ without much pain or no pain.

We concluded from the patient VPS (verbal pain score) rating that there no pain felt during the lip augmentation procedure with the topical anesthetic use. Most patients felt very comfortable and recovered from the freezing effects within 60-75 minutes after the applications. There were no adverse reactions associated in this trial with topical anesthetic. Compared to LMX-4 our topical anesthetic cream was very high by patients as shown in the above FIGURE (VAS pain score).

### Evaluation of a Trans-Oral Delivery System for Topical Anesthesia

Fear of needles arising from pain induced by needle sticks, or Ns, that occur when oral tissues are being anesthetized is a major deterrent to dental care for many patients. A common practice used to prevent such pain is the application of topical anesthetic to the tissue site before Ns. The reported effectiveness of this procedure, however, is controversial and questionable. Variables associated with this practice of topical anesthesia include the composition of the anesthetic; the application medium; the absorption rate; and the patient's psychological perception of pain, the pain threshold or both.

A new topical delivery system that effectively anesthetizes oral tissues may prove highly useful in allaying patient anxieties about and fear of select dental procedures.

The development of FATAC has allowed for the topical delivery of a variety of medicaments, including anesthesia, and for the reduction of pain. benzocaine-tetracaine based anesthetic has been incorporated into this nove gel formulation, which delivers the anesthetic trans-orally. The anesthesia is absorbed within five minutes when applied directly to the oral mucosa. Maximum effect is reached within 15 minutes and has a duration as long as 75 minutes.

We have evaluated a trans oral delivery system for topical anesthesia and report on its efficacy compared with a commonly used gel containing anesthetic in reducing reduction caused by Ns and scaling

### Materials and Methods

We recruited three groups of 20 adults from the patient pool at The Argentina University College of Dentistry. In accordance with our inclusion criteria, we accepted as subjects men and women older than 19 years and younger than 70 years who possessed an adjacent molar-premolar tooth pair in the maxillary right and left quadrants. Subject exclusion criteria included systemic conditions such as cardiovascular diseases, diabetes, mental in competencies, allergic reactions to lidocaine and/or the use of drugs precluding the use of epinephrine, as well as women who were pregnant, nursing or attempting to conceive. Before participating in the study, all of the subjects were required to sign a consent form that had been approved by the university's institutional review board.

A benzocaine-tetracaine-containing gel, supplied at 18% benzocaine-2% tetracaine concentration. For comparative testing, we chose a commonly used topical benzocaine-containing gel, or B-G, anesthetic preparation (Hurricaine, Beutlich, Waukegan, Ill.). Before applying any test or control material, we air-dried the designated tissue sites for 30 seconds with an air-water syringe to remove mucous and saliva. We used each material according to its manufacturer's written directions and removed it before applying any of the clinical stimuli. Briefly, the manufacturer's directions to apply it to the mucogingival tissues for 15 minutes, while those for B-G were to apply a “small amount” for 30 seconds. To ensure maximum absorption, we applied B-G for one minute. After application, we removed both materials and wiped clean the specific tissue sites with a gauze square to remove traces of the material before Ns and Sc/RP instrumentation

Results. Paired t tests and signed ranked tests revealed that the subjects' perception of pain was significantly reduced after the application of FATAC with placebo (P<0.0001) for both Ns and Sc/RP. FATAC also significantly reduced the subjects' perception of pain caused by Ns and Sc/RP when compared directly with B-G (P<0.001). The resultant tissue anesthesia by FATAC significantly reduced pain to Ns with or without anesthetic injection using 25- and 27-gauge needles. However, Ns in conjunction with anesthetic injections generated significantly greater pain than that caused by Ns alone (P<0.01). VPS (verbal pain score) score differences between 25- and 27-gauge needles were not found.

Conclusions. This study found that FATAC caused highly effective anesthesia in alleviating pain/discomfort arising from needle sticks and during the dental procedures and was preferred by all subjects.

Clinical Implications. A new topical delivery system that effectively anesthetizes oral tissues may prove highly useful in allaying patient anxieties about and fear of select dental procedures.

Although the description of the invention identifies a delivery system for topical anesthetic as a primary beneficiary of the invention, it is not limited thereto. It is applicable to other materials and methodologies to permit the patient to achieve the desired result. Those skilled in the art will recognize that there exist a substantial number of variations that could be used in conjunction with one or more aspects of the invention and that the invention could be implemented with different active ingredients. While the above is a description of specific embodiments of the invention, numerous additional embodiments are possible. Moreover, various aspects of the invention may be modified, combined, taken in varying order, added to or taken out without departing from the spirit and breadth of the invention. Similar pathways and equivalent means and steps may be employed within the scope of the inventive concept. Therefore, the above descriptions should not be taken as in any way limiting the scope of invention.

It is to be understood that the above-described compositions and methods are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in materials, temperature, function, order, and manner of operation, assembly and use may be made without departing from the principles and concepts set forth herein.

What is claimed is:

1. A nano particle drug delivery system for topical administration of a local anesthetic agent comprising:
a. a local anesthetic agent;
b. a micelle for encapsulating the local anesthetic agent; and,
c. a lipid molecule layer coating substantially all of each micelle

2. A nano particle drug delivery system according to claim 1, wherein the micelles coated with lipid molecules non-invasively deploy the local anesthetic agent through the stratum corneum and throughout the skin and sub cutaneous tissue without any cutaneous toxicity.

3. A nano particle drug delivery system according to claim 1, wherein the micelles coated with lipid molecules permit the local anesthetic agent to be absorbed through the stratum corneum and throughout the skin and sub cutaneous tissue without any cutaneous toxicity.

4. A nano particle drug delivery system according to claim 1, wherein the local anesthetic agent is Lidocaine.

5. A nano particle drug delivery system according to claim 1, wherein the local anesthetic agent is a combination of Lidocaine and one or more other Caine anesthetic agents.

6. A nano particle drug delivery system according to claim 1, wherein the local anesthetic agent is a combination of one or more other Caine anesthetic agents.

7. A nano particle drug delivery system according to claim 1, wherein the local anesthetic agent is added to alcohol mixtures.

8. A nano particle drug delivery system according to claim 7, wherein the alcohol mixtures are comprised of ethyl, isopropyl, propylene glycol, cetyl and certery alcohol, and lanolin alcohol.

9. A nano particle drug delivery system according to claim 7, wherein after the local anesthetic agent is added to the alcohol mixtures it is added to a solution to form micelles.

10. A nano particle drug delivery system according to claim 9, wherein the solution is Phase-A.
